(19) United States
(12) Patent Application Publication
(10) Pub. No.: US 2025/0090061 A1
(43) Pub. Date: Mar. 20, 2025

(54) Single Use Lancing Device

(71) Applicant: Owen Mumford Limited, Woodstock (GB)

(72) Inventors: Matthew David Farmer, Woodstock (GB); Luke Michael Bailey, Woodstock (GB)

(21) Appl. No.: 18/884,657

(22) Filed: Sep. 13, 2024

(30) Foreign Application Priority Data
Sep. 15, 2023 (GB) 2314129.4

(51) Int. Cl. A61B 5/151

(52) U.S. Cl. CPC A61B 5/15144

(57) ABSTRACT

A lancing device for use in producing a sample of blood on a subject's skin. The device comprises a housing, a lancet comprising a needle having a needle tip, lancet retaining features for releasably retaining the lancet in a forward position prior to use, a shroud coupled movably to the housing and configured to cover the needle tip when the lancet is in the forward position and a resiliently deformable member coupled between the shroud and the lancet. The shroud is configured to, when pressed against the subject's skin, move rearwards relative to the housing whilst compressing the resiliently deformable member, and the lancet retaining features are configured to, at a point of the rearward movement of the shroud, release the lancet allowing said member to expand and exert a rearward force to retract the lancet into the housing.

### CROSS-REFERENCE TO RELATED APPLICATIONS

The present application claims priority to and the benefit of United Kingdom Patent Application Serial No. GB 2314129.4, filed Sep. 15, 2023, and entitled, “Single Use Lancing Device,” the contents of which are incorporated herein by reference in their entirety.

### TECHNICAL FIELD

The present disclosure relates to single-use lancing devices, for example for use in providing a sample of blood.

### BACKGROUND

Lancing devices are used to obtain blood samples from a person or animal by piercing the skin on a finger or other body part with a small needle or blade. Such blood samples are required for many medical tests, for example glucose tests for diabetes management. To make the process of drawing the blood sample convenient and safe, lancing devices need to satisfy high standards. For example, in order to avoid infection and contamination, lancing devices are preferably single-use disposable products. They must therefore be small and simple, to be economic, and preferably should use a sustainable design to minimise waste and environmental impact through their life cycle.

Disposable lancing devices typically include a needle connected to a holder, collectively referred to as a lancet, and a trigger mechanism. When the trigger is activated the lancet is propelled from a retracted position within the housing of the device to an extended position wherein the needle projects from the housing to prick the subject's skin. Usually the lancet is driven by a metal spring which is cocked during the assembly of the device and released by the trigger. A problem with these devices is that the use of metal springs negatively contributes to the device's environmental impact during disposal. The use of a metal spring may also dictate the properties of other, non-metal components, often making the requirements for strength and durability more stringent.

### SUMMARY

According to a first aspect there is provided a lancing device for use in producing a sample of blood on a subject's skin. The device comprises a housing, a lancet comprising a needle having a needle tip, lancet retaining features for releasably retaining the lancet in a forward position prior to use, a shroud coupled movably to the housing and configured to cover the needle tip when the lancet is in the forward position and a resiliently deformable member coupled between the shroud and the lancet. The shroud is configured to, when pressed against the subject's skin, move rearwards relative to the housing whilst compressing the resiliently deformable foam material, and the lancet retaining features are configured to, at a point of the rearward movement of the shroud, release the lancet allowing said member to expand and exert a rearward force to retract the lancet into the housing. The resiliently deformable member may be of a foam material (e.g. a closed-cell or open-cell foam).

Embodiments of the present invention may enable, for example, disposable lancing devices which do not require (metal) springs but still provide automatic needle retraction (after use of lancing device). Embodiments may achieve this by providing a resiliently deformable member which is compressed during use (i.e. during an initial skin pricking phase) and which is released to drive the needle of the device rearward into the housing of the device once a predetermined skin penetration depth is reached. Embodiments of the present invention may thereby enable more sustainable designs of disposable lancing devices. Further, compared to conventional, spring driven devices, embodiments may provide a slower retraction of the needle tip which may lessen tissue damage and pain—this is because the needle tip is typically damaged, i.e. deformed, from pricking the skin and fast retraction of such damaged needle tip can result in substantial tissue damage and pain.

In an embodiment, the lancet retaining features may comprise one or more deflectable fingers extending from an inner surface of the housing, and one or more shoulders provided on the lancet are configured to engage with the or each deflectable finger to restrict a rearward movement of the lancet until a force exerted by the shroud causes sufficient deflection of the or each finger. The or each deflectable finger may extend forward from a rearward end of the housing. The or each deflectable finger may comprise, at a forward end thereof, a hook portion protruding towards a central axis of the housing for engaging with a shoulder of the lancet.

In an embodiment, the lancet retaining features may be configured, upon release of the lancet, to further urge the lancet in a rearward direction. The further urging of the lancet in a rearward direction may be facilitated by cooperating inclined and opposed surfaces of the or each finger of the lancet retaining features and the or each shoulder of the lancet. The further urging of the lancet may facilitate an initial phase of the needle retraction which can require a high force to “break loose”.

In an embodiment, prior to use, the member may be held in a partially compressed state so as to bias the lancet rearward and thereby engage the lancet retaining features. This may prevent, for example, a rattling noise when the device is handled.

In an embodiment, the member may be tubular and surrounds a portion of the lancet. Such a member may provide a good ratio of provided force to required space.

In an embodiment, the shroud may be configured to directly contact the lancet prior to the point of the rearward movement of the shroud so as to exert a rearward force on the lancet in addition to the rearward force exerted by the member. This makes it possible to precisely control the skin penetration depth.

In an embodiment, the shroud may be slidably received within the housing and being held in an extended position by cooperating features of the shroud and the housing. The cooperating features may provide a resistance force when the shroud is pressed against the skin that must be overcome before the shroud can be moved into the housing from the forward position. This may have the effect that an accidental activation of the lancing device is prevented.

### BRIEF DESCRIPTION OF THE DRAWINGS

FIG. 1 shows a front view of an assembled lancing device according to an embodiment;

FIGS. 2A and 2B are exploded (front and perspective) views of the lancing device of FIG. 1;

FIGS. 3A-D show respectively perspective views of a shroud, a lancet, a cap and a housing of the lancing device of FIG. 1;

FIG. 4A shows a cross-sectional front view of the lancing device of FIG. 1 before use;

FIG. 4B shows a cross-sectional front view of the lancing device of FIG. 1 after the cap is removed;

FIG. 4C shows a cross-sectional front view of the lancing device of FIG. 1 in use where a needle is inserted into the skin; and

FIG. 4D shows a cross-sectional front view of the lancing device of FIG. 1 after usage.

### DETAILED DESCRIPTION

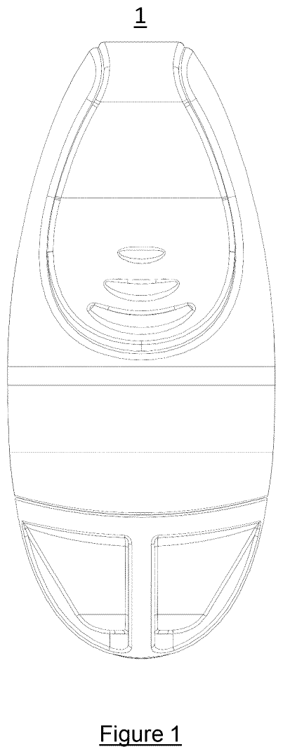

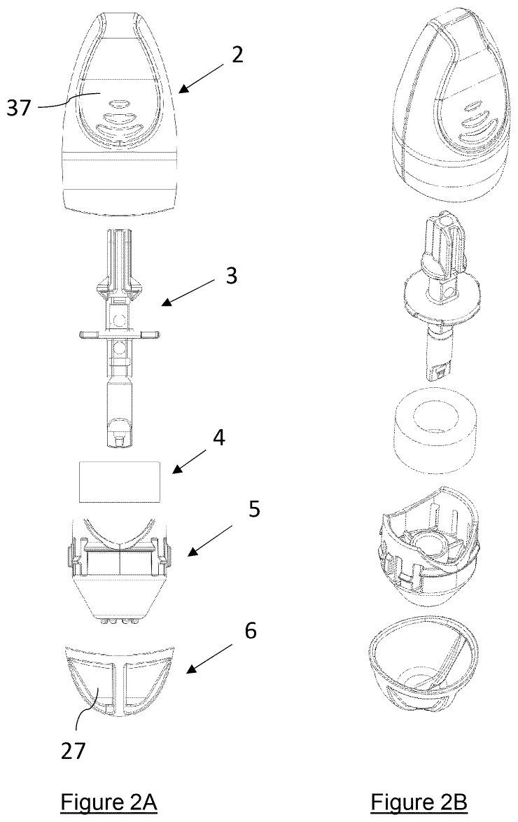

The accompanying Figures illustrate the components and operation of a single use lancing device designed for pricking a subject's skin to, for example, provide a blood sample. In the following discussion a human subject is considered although it will be appreciated that the device may be used for an animal subject. An embodiment is described with reference to FIGS. 1-4. The references to “lower”, “upper”, “horizontal” and “vertical” have been chosen for convenience, and refer to the orientations as shown in FIGS. 1, 2A and 4A-D. Referring to FIG. 1, a lancing device 1 is shown in an assembled state. As further illustrated in the exploded views of FIGS. 2A and 2B, the lancing device 1 has a housing 2, a lancet 3, a resiliently deformable foam member 4, a shroud 5, and a cap 6.

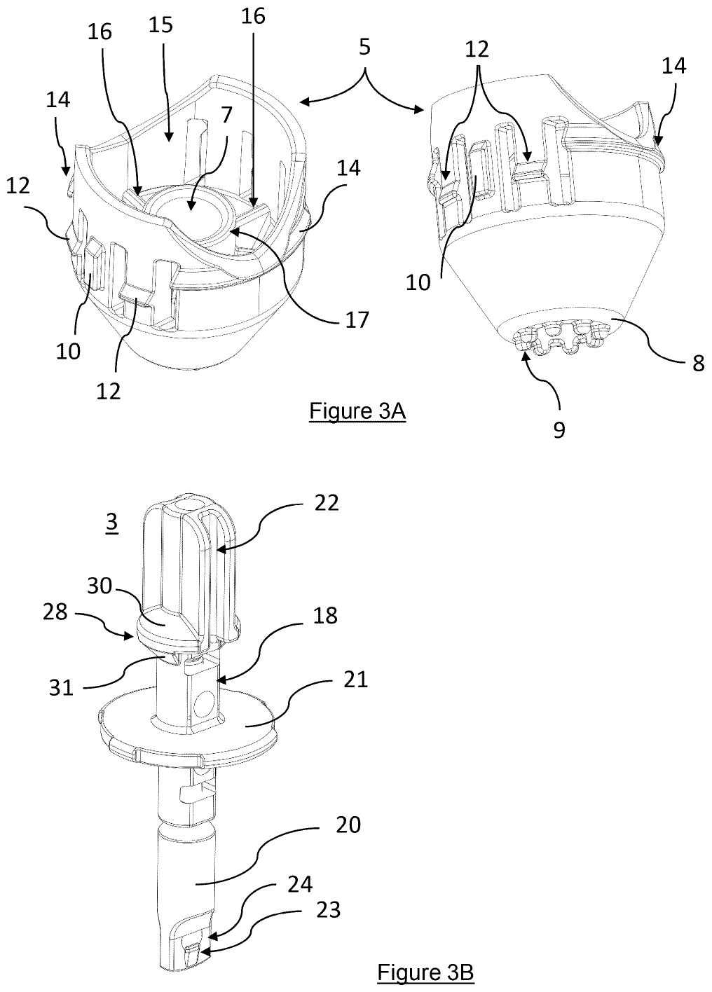

Referring to FIG. 3A, the shroud 5 comprises a generally tubular shaped section and provides an axial passage 7. The lateral extent of the shroud 5 is greater at its upper end than at its lower end. The lower end of the shroud 5 has a generally ring-shaped skin contacting surface 8. Projections 9 on the skin contacting surface are intended to “confuse” the nerves in the area of the prick to make the prick less painful to the user. The shroud 5 comprises features to prevent rotation relative to the housing 2 when assembled. In particular, the shroud 5 comprises two ribs 10 which extend laterally from an outer surface of the shroud 5. The ribs 10 are configured to cooperatively engage with grooves 11 in the housing 2, preventing rotational movement of the shroud 5 relative to the housing 2. The shroud 5 also provides four deflectable fingers 12 on its outer surface. Vertically elongated cut-outs radially adjacent to the fingers 12 ease inward deflection of the fingers 12 (i.e. deflection toward a central axis of the shroud 5). As described further below, the fingers 12 are configured to cooperatively engage with protrusions 13 of the housing 2 to prevent accidental activation of the lancing device. Both perspective views provided in FIG. 3A show only one rib 10 and two fingers 12 as the second rib and the other two fingers are located on an opposite side of the housing 2 and is not visible in these views. The shroud 5 further provides a protrusion 14 extending from the outer surface of the shroud 5. The protrusion 14 is generally annular shaped. Instead of forming a complete ring, the protrusion 14 may comprise two portions, which are arc shaped, as illustrated in FIG. 3A.

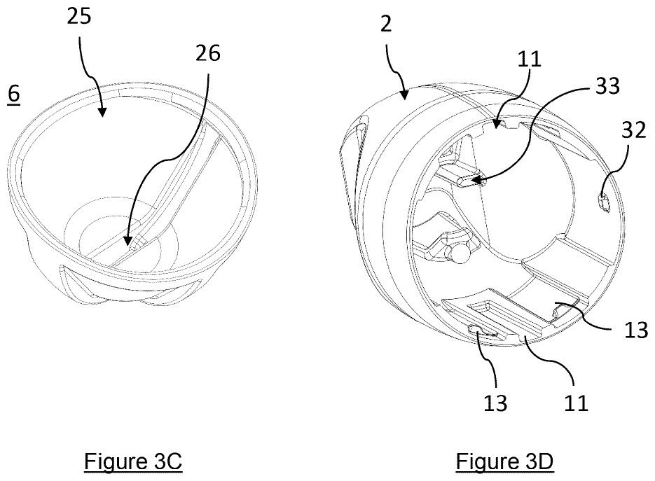

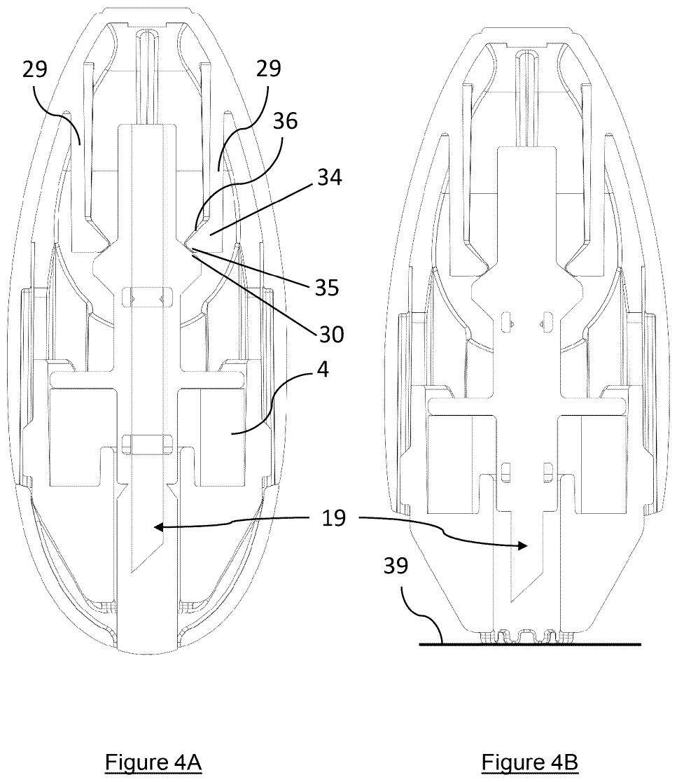

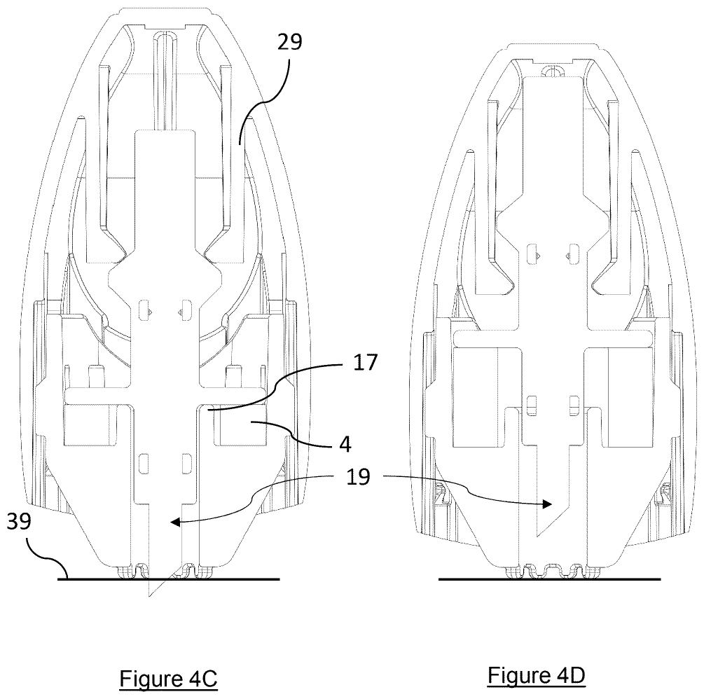

At an upper end, the shroud 5 further defines an opening 15 for receiving the foam member 4. The shroud 5 further includes an upward facing surface for supporting a lower end of the foam member 4. In the embodiment of FIG. 3A, this supporting surface is provided by an inner structure 16 comprising eight walls (only two are visible in FIG. 3A) extending from an inner surface of shroud 5 (fewer or more than eight walls may also be envisaged). Within the opening 15, the passage 7 is defined by a cylindrical structure 17. The surface for supporting the lower end of the foam member 4 is lower than an upper surface of the cylindrical structure 17. As described below with reference to FIG. 4C, this allows precise control over the maximum skin penetration depth.

The resiliently deformable foam member 4 is generally cylindrically-shaped with a central aperture along its longitudinal axis. The outer diameter of the foam member 4 is small enough so that the foam member 4 can be at least partially received by the opening 15 of the shroud 5. The inner diameter of the foam member 4 is larger than the outer diameter of the cylindrical structure 17. In other embodiments, the foam member 4 may have a different shape (i.e. not cylindrically-shaped). For example, the foam member may have (in a horizontal cross-section) a square (or hexagonal) shaped outer profile and a round aperture in the centre. A cylindrically-shaped foam member may be particularly well suited to the lancing device 1 since such a member provides a good ratio of provided force to required space.

Referring to FIG. 3B, the lancet 3 comprises an elongated body 18, and a needle 19 embedded within the body 18 and initially shrouded by a cover 20. The needle 19 (not visible in FIG. 3B) is visible in FIGS. 4A-D. The body 8 has a laterally extending lower shoulder 21 which provides generally flat and horizontal surfaces at its lower and upper ends. At an upper end, the body 18 comprises two vertically extending grooves 22. The grooves 22 are configured to receive ribs 3 extending from an inner surface of the housing 2 so as to prevent rotation of the lancet 3 around its central axis.

The lancet 3 comprises features for engagement with the housing 2 to control the retaining and retraction of the lancet 3 during use of the device. To this end, the lancet 3 comprises a laterally extending upper shoulder 28 provided above the lower shoulder 21, as illustrated in FIG. 3B. Generally, the upper shoulder 28 is configured to engage with two deflectable fingers 29 of the housing 2. In particular, at its upper end, a portion of the upper shoulder 28 is tapered towards a central axis of the lancet 3 providing an upper inclined surface 30. Similarly, at its lower end, a portion of the upper shoulder 28 is tapered towards the central axis of the lancet 3 providing a lower inclined surface 31, for example at an angle of inclination of about 45°.

Referring to FIG. 3B, the cover 20 is moulded integrally with the body 18, but the cover 20 can be twisted off to expose the needle tip extending from a lower end of the body 18, i.e. an initial connection between the cover 20 and the body 18 is sufficiently weak that twisting of the cover 20 relative to the body 18 breaks the cover 20 free without damaging the body 18. The cover 20 comprises features that allow the cover 20 to be attached to the cap 6. In particular, a lower portion 24 of the cover 20 is provided which is generally flat and wide in shape, and is configured to engage with a pocket 26 of the cap 6. The lower portion 24 of the cover 20 provides two hooks 23 (only one hook is visible in FIG. 3B) configured to cooperatively engage with corresponding recesses in the pocket 26 of the cap 6 to attach the cover 20 to the cap 6.

Referring to FIGS. 1, 2 and 3C, the cap 6 is closed at its lower end and defines a generally circular opening 25 at its upper end. The cap 6 comprises the pocket 26 configured to receive at least a portion of the lower portion 24 of the cover 20. The pocket 26 comprises recesses configured to cooperatively engage the hooks 23 on the lower portion 24 of the cover 20 to (non-releasably) attach the cover 20 and the cap 6. The cap 6 further comprises four grip areas 27 to assist a user in grasping the cap 6 to facilitate rotation of the cap 6 relative to the housing 2 in order to twist off the cover 20 as described further below. FIG. 1 shows only two grip areas 27 as the other two grip areas are located on an opposite side of the cap 6 and are not visible in this view. It will be understood that, in embodiments, the outer surface of the cap 6 may be formed differently from the embodiment shown in the Figures, although it is desirable that the outer surface is structured to allow a user to rotate the cap 6 relative to the housing 2: for example fewer or more than four grip areas may be provided.

Referring to FIGS. 2A and 3D, the housing 2 is closed at its upper end and defines a generally circular opening at its lower end. On an outer surface, the housing 2 provides two grip areas 37 to assist a user in grasping the device during operation. FIG. 2A shows only one grip area 37 as the second grip area is located on an opposite side of the housing 2 and is not visible in this view. The opening of the housing 2 is configured to receive an upper portion of the shroud 5. The housing 2 comprises two grooves 11 on its inner surface which are configured to receive the ribs 10 of the shroud 5. The housing further comprises four protrusions 13 configured for engagement with the corresponding four fingers 12 of the shroud 5. Further, the housing 2 comprises two bumps or lugs 32 (only one lug 32 is visible in FIG. 3D, the other one in on the opposite side). The lugs 32 are configured to engage the protrusion 14 of the shroud 5 to restrict forward movement of the shroud 5 with respect to the housing 2 (in an assembled device). The housing 2 also comprises two ribs 33 extending from an inner surface of the housing 2. The ribs 33 are configured to be received by the grooves 22 of the lancet to restrict rotational movement of the lancet 3.

The housing 2 further comprises features for engagement with the lancet 3 to control the retaining and retraction of the lancet 3 during use of the device. In particular, the housing 2 comprises the two deflectable fingers 29, as shown in the cross-sectional view of FIG. 4A. The deflectable fingers 29 extend downwards from an inner surface of the housing 2. Each of the fingers 29 has at a lower end thereof a hook portion 34 protruding inwards (i.e. towards a central axis of the housing 2). Each hook portion 34 provides a lower inclined surface 35 arranged to abut the upper inclined surface 30 of the upper shoulder 28 of the lanced 3 in an assembled state of the lancing device 1. Further, each hook portion 34 provides an upper inclined surface 36 arranged to contact the lower inclined surface 31 of the upper shoulder 28 of the lanced 3 during use of the lancing device 1, as described in more detail below.

The housing 2, the shroud 5, the lancet body 18 and the cap 6 are moulded components. Considering the lancet 3, the needle 19 is embedded into the lancet body 18 by over-moulding. The foam member 4 may be made from any suitable solid foam. For example, the foam member 4 may be made from open-cell or closed cell foam. The foam members may be made from closed-cell foam which is particularly well suited for use in the compact lancing device 1 since these members provide enough force to reliably retract the lancet 3 (when released from a compressed state). Further, the foam may be made from any suitable material, for example plastic. In particular, the foam may be a low density foam. In one embodiment, the density of the foam may be 15 kg/m³.

In general, it is understood that foam is only one of many resiliently deformable materials which are suitable for use in the proposed lancing device. In other embodiments, the member 4 may be made of a resiliently deformable material other than foam.

The assembly of the lancing device 1 comprises four simple steps. First, the foam member 4 is inserted into opening 15 of the shroud 5 from the upper end of shroud 5. Second, the lancet 3 (sterilised prior to assembly) is installed by inserting it into the passage 7 from the upper end of the shroud 5 so that the lower surface of the lower shoulder 21 of the lancet 3 contacts the upper end of the foam member 4. At this stage, the lower portion 24 of the lancet cover 20 extends outwardly from the passage 7 of the shroud 5. Third, the cap 6 is connected to lancet 3 by inserting the lower portion 24 of the lancet cover 20 into the pocket 26 of the cap 6. Fourth, the housing 2 is connected to the shroud 5 by pushing the upper end of the shroud 5 into the open, lower end of the housing 2 until the protrusion 14 on the shroud 5 rides over the lugs 32 of the housing 2 securing the shroud 5 to the housing 2.

In an assembled lancing device, the ribs 10 of the shroud 5 are located in the grooves 11 of the housing 2 to prevent rotation of the shroud 5 and the housing 2 in relation to each other. Similarly, the ribs 33 are located in the grooves 22 of the lancet 3 to prevent rotation of the lancet 3 and the housing 2 in relation to each other. In the assembled state, as shown in FIG. 4A, the lancet 3 is retained in an operating position. The upper shoulder 28 abuts the fingers 29, restricting a rearward movement of the lancet 3 into the housing 2. In particular, the upper inclined surface 30 of the lancet 3 is in contact with the lower inclined surface 35 of the fingers 29. A forward movement of the lancet 3 is restricted as its lower shoulder 21 is in contact with the foam member 4. In the assembled state, the foam member 21 is slightly compressed, thus exerting an upward force onto the lancet 3. The lancet 3, in turn, exerts this upward force onto (the hook portions 34 of) the fingers 29. The fingers 29 are stiff enough so that this force is too low to (substantially) deflect the finger 29. It is an advantage that the foam member is slightly compressed in the assembled state of the lancing device since this secures the lancet and prevents, for example, a rattling noise when the device is handled.

With reference to FIGS. 4A to 4D, the operation of the lancing device will now be described. In order to prepare the lancing device 1 for use, a user holds the lancing device 1 with the fingers of one hand by the grip areas 37, grips the cap 6 with the fingers of the other, and twists the cap 6 (and thereby the lancet cover 20). This action breaks the connection between the lancet body 18 and the lancet cover 20 allowing the lancet cover 20 (connected to the cap 6) to be pulled off the (sterile) needle 19. As the ribs 33 of the housing 2 are located in the grooves 22 of the lancet 3, the lancet 3 is prevented from rotating which allows a user to twist the cap 6 relative to the housing 2 to break off the lancet cover 20.

After removal of the cap 6, the user places the skin contacting surface 8 with projections 9 against skin 39 at a sampling site, as illustrated in FIG. 4B. At this stage, the shroud 5 extends forwards beyond a forward end of the needle 19, preventing contact of the skin 39 with needle 19. Rearward movement of the shroud 5 into the housing 2 is initially restricted by an engagement of the fingers 12 with the protrusions 13 of the housing 2. Pressing the lancing device against the skin 39, the user applies a level of force that exceeds a predefined threshold force. The predefined threshold force may be between 10-15N. Due to the applied force, the fingers 12 of the shroud 5 are deflected inwardly (i.e. towards the central axis of the device) by the protrusions 13 of the housing 2, allowing the shroud 5 to move rearwards into the housing. At this point, the force, which is still applied by the user, leads to a sudden rearward movement of the shroud 5. During the rearward movement of the shroud 5, the lancet remains retained in its operational position by the fingers 29 of the housing 2, and the foam member 4 is further compressed. Upon reaching a first point of the rearward movement of the shroud 5, the needle 19 contacts the sampling site.

The user then continues to press the lancing device 1 against the skin 39. The shroud 5 moves further rearwards, and the needle 19 further penetrates the skin 39, and the foam member 4 is further compressed. As illustrated in FIG. 4C, at a second point of the rearward movement of the shroud 5, the needle 19 reaches a predetermined skin penetration depth. At this stage, the foam member 4 is compressed sufficiently that the shroud 5 comes into direct contact with the lancet 3. In particular, the upper end of the cylindrical structure 16 of the shroud 5 abuts the lower shoulder 21. The fingers 29 are not (substantially) deflected at this stage. The force exerted by the foam member 4 at this stage may be less than 6N. Generally, the provision of direct coupling between the shroud 5 and the lancet 3, as described above, allows precise control over the skin penetration depth.

The applied force overcomes the resistance of the fingers 29 which start to flex. Thus, the upper inclined surface 30 of the upper shoulder 28 is pushed against the lower inclined surface 35 of the fingers 29 lower to flex the fingers outwardly (i.e. away from the central axis of the device). As the fingers 29 flex, both the shroud 5 and the lancet 3 move rearwards into the housing 2. Since the shroud 5 and the lancet 3 move together, the lancet 3 still penetrates the skin 39 with the predetermined skin penetration depth.

Next, the shroud 5 reaches a point during the rearward movement when the fingers 29 (riding over the upper shoulder 28 of the lancet 3) are maximally deflected. The fingers 29 are now flexed away from the central axis of the housing 2 and further rearward movement of the lancet 3 is no longer restricted within the housing 2 and the energy stored in the foam member 2 is released to drive the lancet 3 from the operational position to a retracted position. In other words, the foam member 4 decompresses and pushes the lancet 3 rearwards towards the retracted position. The lancet 3 is further pulled rearwards (i.e. towards the retracted position) by finger 29. As the fingers 29 flex back, the lower inclined surface 31 of the lancet 3 contacts the upper inclined surface 36 of the hooks 34 pulling the lancet 3 rearwards. It is advantageous that the fingers 29 provide an additional rearward force to the lancet 3 because this lowers the force requirements for the foam member 4. During an initial phase of the needle retraction a high force is typically required to “break loose”. This can be because the needle tip is deformed (e.g. into a hook-like shape) during the initial skin penetration causing high resistance against rearward movement. Because the fingers 29 provide enough rearward force for the initial break loose of the lancet 3, the foam member 4 only needs to provide enough force to complete the retraction into the housing. Further, because the foam member 4 can release the retraction force onto the lancet 3 more slowly compared to conventional springs, the needle 19 is retracted from the skin 39 at a slower speed which can reduce tissue damage and pain to the user.

As shown in FIG. 4D, when the lancet 3 is in the retracted position, the needle 19 does not extend outwardly from the passage 7 of the shroud 5. Next, the user removes the lancing device from the sampling site and collects the blood sample, e.g. with a suitable swab. As the lancet 3 is now in the retracted position, the risk of accidental needle injuries is greatly reduced. The lancing device is now in a “used” state and can be disposed of. The removed cap 6 and retracted shroud 5 serve as a visual indicator to the user that the device has been used and thereby prevents any confusion by the user as to the state of the lancing device.

The lancing device 1 described with respect to the accompanying figures provides a very simple and elegant solution to the problem of manufacturing disposable lancing devices that provide a consistent and well-defined skin penetration depth but do not require (metal) springs. The simplicity of the described lancet retraction mechanism reduces the points of failure which may increase reliability of the device and improve the ease of manufacturing.

Although the present invention has been described with reference to a foam material, the skilled person would understand that any alternative resiliently deformable member may be used. For example, rubber or an air filled circular pocket may be used instead of foam.

Those skilled in the art will appreciate that various modifications may be made to the above described embodiment without departing from the scope of the present invention.

In the above description, the pricking member that pierces the skin has been described as a “needle”. Those skilled in the art will appreciate that this term encompasses conventional needles, blades etc.

| Reference numeral | Feature |
|---|---|
| 1 | Lancing device |
| 2 | Housing |
| 3 | Lancet |
| 4 | Foam ring |
| 5 | Shroud |
| 6 | Cap |
| 7 | Shroud passage |
| 8 | Skin contacting surface |
| 9 | Projections |
| 10 | Ribs |
| 11 | Grooves |
| 12 | Fingers |
| 13 | Protrusions on housing |
| 14 | Protrusion on shroud |
| 15 | Upper opening |
| 16 | Inner structure |
| 17 | Cylindrical structure |
| 18 | Lancet body |
| 19 | Needle |
| 20 | Lancet cover |
| 21 | Lancet shoulder |
| 22 | Lancet grooves |
| 23 | Lancet cover hooks |
| 24 | Lower portion of lancet cover |
| 25 | Opening in cap |
| 26 | Pocket in the cap |
| 27 | Grip area cap |
| 28 | Upper shoulder of lancet |
| 29 | Deflectable fingers of the housing |
| 30 | Upper inclined surface |
| 31 | Lower inclined surface |
| 32 | Lugs on housing |
| 33 | Ribs on the housing |
| 34 | Hook portion |
| 35 | Lower inclined surface of deflectable finger |
| 36 | Upper inclined surface of deflectable finger |
| 37 | Grip area on housing |
| 38 | Skin |

1. A lancing device for use in producing a sample of blood on a subject's skin, the device comprising:
a housing;
a lancet comprising a needle having a needle tip;
lancet retaining features for releasably retaining the lancet in a forward position prior to use;
a shroud coupled movably to the housing and configured to cover the needle tip when the lancet is in the forward position; and
a resiliently deformable member coupled between the shroud and the lancet,
wherein the shroud is configured to, when pressed against the subject's skin, move rearwards relative to the housing whilst compressing the resiliently deformable member, and the lancet retaining features are configured to, at a point of the rearward movement of the shroud, release the lancet allowing said member to expand and exert a rearward force to retract the lancet into the housing.

2. A lancing device according to claim 1, wherein the resiliently deformable member comprises a closed-cell or open-cell foam.

3. A lancing device according to claim 1, wherein the lancet retaining features comprise one or more deflectable fingers extending from an inner surface of the housing, and wherein one or more shoulders provided on the lancet are configured to engage with the or each deflectable finger to restrict a rearward movement of the lancet until a force exerted by the shroud causes sufficient deflection of the or each finger.

4. A lancing device according to claim 3, wherein the or each deflectable finger extends forward from a rearward end of the housing.

5. A lancing device according to claim 3, wherein the or each deflectable finger comprises, at a forward end thereof, a hook portion protruding towards a central axis of the housing for engaging with a shoulder of the lancet.

6. A lancing device according to claim 1, wherein the lancet retaining features are configured, upon release of the lancet, to further urge the lancet in a rearward direction.

7. A lancing device according to claim 3, wherein the lancet retaining features are configured, upon release of the lancet, to further urge the lancet in a rearward direction and said further urging of the lancet in a rearward direction is facilitated by cooperating inclined and opposed surfaces of the or each finger and the or each shoulder.

8. A lancing device according to claim 1, wherein, prior to use, said member is held in a partially compressed state so as to bias the lancet rearward and thereby engage the lancet retaining features.

9. A lancing device according to claim 1, wherein the member is tubular and surrounds a portion of the lancet.

10. A lancing device according to claim 1, wherein the shroud is configured to directly contact the lancet prior to said point of the rearward movement of the shroud so as to exert a rearward force on the lancet in addition to the rearward force exerted by the member.

11. A lancet device according to claim 1, the shroud being slidably received within the housing and being held in an extended position by cooperating features of the shroud and the housing, the cooperating features providing a resistance force when the shroud is pressed against the skin that must be overcome before the shroud can be moved into the housing from the forward position.
